(19) United States
(12) Patent Application Publication
(10) Pub. No.: US 2007/0073185 A1
(43) Pub. Date: Mar. 29, 2007

(54) Needle biopsy forceps with integral sample ejector

(76) Inventor: Naomi L. Nakao, New York, NY (US)

Correspondence Address: COLEMAN SUDOL SAPONE, P.C., 714 COLORADO AVENUE, BRIDGE PORT, CT, 06605-1601, US

(21) Appl. No.: 10/556,543

(22) PCT Filed: Mar. 3, 2005
(86) PCT No.: PCT/US05/07340

§ 371 Date: Nov. 14, 2005

(51) Int. Cl. A61B 10/00; A61B 17/00

(52) U.S. Cl. 600/564; 606/205; 606/206

(57) ABSTRACT

A needle biopsy instrument for retrieving a tissue sample from an internal organ such as needle biopsy forceps, includes a pushing surface for safely dislodging a tissue sample from the needle after the instrument containing the sample is retrieved from the patient using mechanical control means operated from the proximal handle to thereby avoid the risk of accidental cuts, punctures and other injuries to medical personnel while trying to manually remove the sample. The instrument has a cam mechanism, or other mechanical links scissor links for operating the cups and producing relative movement between the pushing surface and axis of the needle to cause a tissue sample to be moved toward and eventually off of the tip of the needle into a sample receiving cup. An axial reciprocating control member attached to either the needle or the pushing surface produces the relative movement and is manually controlled by a handle or other grip at the proximal end of the forceps. The needle is either retracted axially from the distal toward the proximal end of the device, or a pushing member attached to the distal end of an actuating cable or cable is moved distally along the axis of a stationary needle. A latch mechanism can be provided on the proximal end to release a biasing force to provide the relative movement between the needle and pushing member to slide a tissue sample off the needle.

### FIELD OF THE INVENTION

The invention relates to flexible biopsy forceps used in conjunction with a flexible endoscope and rigid forceps used with a laparoscope for retrieval of a tissue sample from the interior of a patient's body, where the forceps include a needle that passes through the tissue to be sampled in order to retain one or more severed samples for retrieval.

### BACKGROUND OF THE INVENTION

Flexible needle biopsy forceps are used in conjunction with an endoscope as follows: the endoscope is inserted into a patient's body cavity, an abnormality is visualized, and the biopsy forceps is introduced through the working channel of the endoscope. In the case of a flexible biopsy forceps, the distal end of the biopsy forceps is comprised of two opposed sharp-edged cups that are operably attached by means of pivot arms to a cable passing on the interior of a flexible hollow shaft. A fixed spike or needle is positioned between the cups. As used hereinafter, the term “needle” will be understood to include both a needle and a spike, or other similar member that passes through and retains the severed tissue sample until the forceps are removed from the patient's body. Actuation means, such as thumb and/or finger grips or a spool, are operably connected to the proximal ends of the flexible shaft and the one or more cables are used to move the cups between an open and a closed position.

When the forceps' distal end is properly positioned at the sampling site, the cups are moved to the open position, the needle makes contact with and penetrates the tissue to be sampled and the cups are then closed upon the tissue, grasping and severing a sample of tissue that is held on the needle within the closed cups. One or more samples may be obtained during the same intubation of the biopsy forceps, because the samples are successively held on the spike without falling out of the cups when said cups are opened. Upon withdrawal of the biopsy forceps, the cups are opened and the tissue samples are placed into a preservative solution. In order to dislodge the samples that are stacked on the needle, the operator is required to use a sharp tool to push the samples off of the needle into preservative, thus coming in direct physical contact with the tissue samples, the needle and the sharp device used to dislodge the specimen.

In addition to this task being difficult and cumbersome in itself, it presents the immediate danger to the operator of being injured by the sharp, or the biopsy forceps needle. Many accidents have been reported during which medical personnel have been injured by the sharp. If the patient is infected with the HIV virus, hepatitis, or another contagious disease, the physician or assistant can be infected as well. An additional risk to the medical personnel from an infectious sample is posed by the sharp cutting edges of the cups themselves, which must be maintained in the open position while the tissue sample(s) are removed from the forceps' needle. The rigid forceps that are employed in conjunction with the laparoscopic procedure function in a similar manner, and carry the same risks.

It is therefore desirable to provide an improved needle biopsy forceps that will eject the biopsy sample from the needle or spike into a convenient receptacle by manipulation of interconnected control means at the proximal end of the forceps.

It is further desirable to provide improved needle biopsy forceps from which the tissue sample can be safely ejected without having the medical personnel directly contact or manipulate the distal end of the forceps and which will eliminate the need for such personnel to use needles or other “sharps” to collect the tissue samples.

### BRIEF DESCRIPTION OF THE INVENTION

A needle biopsy instrument for retrieving and ejecting a tissue sample taken from an organ comprises, in accordance with the present invention, (a) an elongate hollow shaft member with a proximal end and a distal end, (b) a needle connected at least indirectly to the shaft member proximate the distal end thereof, the needle being oriented to pass through a portion of tissue to be sampled before the tissue sample is severed from the organ, (c) a tissue ejection element mounted at least indirectly to the shaft member at the distal end thereof, and (d) an ejection mechanism operatively connected to at least one of the needle and the tissue ejection element for moving the needle and the tissue ejection element relative to one another so that the tissue ejection element engages or contacts the tissue sample and the tissue sample is dislodged from a distal end of the needle.

As broadly contemplated, the working, or distal end of a sample ejecting needle biopsy forceps assembly in accordance with the present invention includes a tissue sample contacting surface located at the proximal end of the needle and means for producing relative movement between this surface and the needle to thereby contact the sample and push it along the longitudinal axis of the needle when forceps cups are in the open position. This allows the operator to safely and precisely deposit the sample in a preservative container for later analysis. The relative movement is accomplished by a control mechanism located at the proximal end of the forceps assembly. The control mechanism comprises the handle or, alternatively, is positioned near the handle. At least one linking member is slidably disposed in a hollow shaft and extends from the control mechanism in the handle to the distal end of the instrument.

In one embodiment, the tissue sample contacting surface is located outside of, and proximally displaced from, the cups while the cups are closed and partially opened to sever the tissue sample. Control means at the proximal end of the forceps are manually actuated to provide, via the linking member, relative movement between the needle and the sample contacting surface to contact and dislodge the sample by slidingly advancing the sample to the tip of the needle when the cups are moved to a more fully opened position.

In another embodiment, the tissue sample contacting surface is located between the cups at the juncture of the cup supporting pivot arms, and the contact surface moves along the longitudinal axis of the needle or spike when the cups are fully opened to thereby contact and dislodge the sample.

In a further embodiment, the needle itself is moveable and is attached to a wire linking member and thereby to a control handle located at the proximal end of the forceps. As the wire is withdrawn proximally, so the needle is likewise withdrawn to bring the sample into contact with the contacting surface.

In another embodiment, the sample contacting surface is formed on a longitudinally moveable plate attached to a wire and control means at the proximal end of the forceps. The needle is stationary and the plate moves up the axis of the needle to contact and slide the sample off the tip of the needle.

The above embodiments can also be combined with a fluid reservoir and injection system in communication with a hollow needle and/or a cauterization circuit and controls connected to the cups.

In each embodiment, the sample ejecting means is remotely activated by control means located at the proximal end of the forceps by the axial movement of an axially extending, inextensible, but flexible linking member located in the flexible shaft that is secured to the distal and proximal ends of the forceps assembly. The linking member is comprised of one or more wires, and a coiled wire cable that can be slidably moved within the working channel of an endoscope insertion member or another hollow lumen. The proximal remote control means can be provided with a biasing force that remains armed until manually released.

As used herein, the term “cable” is to be understood to include a single strand or rod, a coiled wire cable made of metal and polymeric materials, or other suitable device.

As will be understood by one of ordinary skill in the art, the method and apparatus of the invention is applicable to, and can be adapted for use with other medical instruments and is not to be construed as limited solely to biopsy forceps.

### BRIEF DESCRIPTION OF THE DRAWINGS

FIG. 1 is a partially cut-away side elevation view of a conventional biopsy forceps pursuant to the prior art;

FIG. 2 is a schematic perspective view of one embodiment of the forceps in accordance with the present invention, in a partially opened position;

FIG. 3a is a partial sectional side view of the distal portion of the forceps depicted in FIG. 2, in a closed position;

FIG. 3b is a partial sectional side view of the distal portion of the forceps depicted in FIG. 2, in a partially opened position;

FIG. 3c is a partial sectional side view of the distal portion of the forceps depicted in FIG. 2, in a fully opened position;

FIG. 3d is a cross-sectional view taken along line 3d-3d of FIG. 3a;

FIG. 3e is a cross-sectional view taken along line 3e-3e of FIG. 3b;

FIG. 3f is a cross-sectional view taken along line 3f-3f of FIG. 3c;

FIG. 4 is a top plan view of a sliding plate member shown in FIG. 3a;

FIG. 5 is a side elevation view of the plate of FIG. 4;

FIG. 6a is an enlarged side elevation view of a portion of spike shown in FIG. 3a;

FIG. 6b is an enlarged side elevation view of the sliding plate member shown in FIGS. 4 and 5;

FIG. 7a is a top plan view of a cup and supporting arm for use in one embodiment of the invention;

FIG. 7b is a side elevation view of the cup of FIG. 7b;

FIG. 8 is a schematic perspective view of biopsy forceps with cups in an open position illustrating another embodiment in accordance with the present invention;

FIG. 9 is an enlarged schematic, partially cut-away and phantom view of the needle reciprocating mechanism for use in the forceps depicted in FIG. 8;

FIG. 10a is a partial sectional view of the distal portion of the forceps depicted in FIG. 8 in a fully open position and ready for use;

FIG. 10b is a partial sectional view of the distal portion of the forceps depicted in FIG. 8 in an open position with the needle partially withdrawn;

FIG. 10c is a partial sectional view of the distal portion of the forceps depicted in FIG. 8 with the needle fully withdrawn from the cups;

FIG. 11 is a plan view in partial section of the distal end of a biopsy forceps illustrating another embodiment of the invention in a closed position;

FIG. 12a is a side view in partial section of the device depicted in FIG. 11 in an open position;

FIG. 12b is a side view in partial section of the device depicted in FIG. 11 in which the ejection means is extended;

FIG. 13 is a front and side perspective view in partial section of the forceps depicted in FIG. 8 illustrating one embodiment of a biased release mechanism;

FIG. 14 is a detail of the mechanism shown in FIG. 13;

FIG. 15A is a front left and top perspective view of the distal end of yet another embodiment of the invention;

FIG. 15B is a partial top plan view of the embodiment depicted in FIG. 15A;

FIG. 16 is a schematic perspective view of a further embodiment in accordance with the present invention having fluid injecting means;

FIG. 17 is a schematic perspective view of another embodiment pursuant to the invention having cauterization capability;

FIG. 18 is a top, front left side schematic perspective view of yet another embodiment in accordance with the present invention;

FIG. 19 is an elevation cross-section view of the embodiment depicted in FIG. 18;

FIG. 20 is a side elevation view, partly in cross-section, of the embodiment depicted in FIG. 18;

FIG. 21 is a schematic perspective view of a further embodiment of the invention illustrating the position of the forceps control and ejection means; and

FIG. 22 is a cross-sectional side elevation view of a portion of the proximal end of the embodiment of the instrument depicted in FIG. 18A schematically illustrating manual control and release members for enhancing the safety aspects of its method of use.

### DETAILED DESCRIPTION

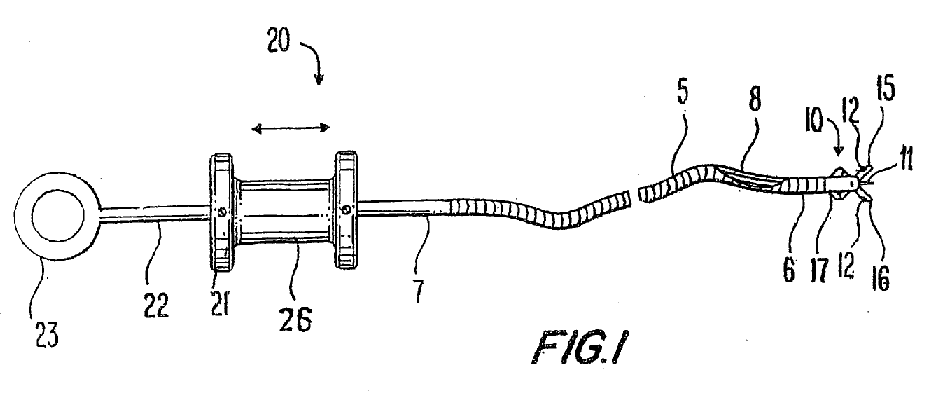

A conventional needle biopsy forceps 1 of a type known in the art is illustrated in FIG. 1. The forceps comprises a hollow flexible shaft 5 having a distal end 6 and a proximal end 7. An actuating cable 8 is slidably disposed within shaft and extends from the distal to the proximal ends of said shaft. At the distal end of the shaft is a tissue sample collection means 10, typically comprising two opposing cups 15 and 16 pivotally attached to a clevis 17 depending from shaft 5. A needle or spike 11 extends from the distal end and is enclosed by cups 15 and 16 when they are in the closed position. The cups are operably attached to cable 8, so that sliding the cable in a distal direction opens the cups, while sliding the cable in a proximal direction closes the cups.

With continuing reference to FIG. 1, there is shown at the proximal end stem 7 of the flexible external shaft 5, and an actuation handle mechanism 20 typically comprising a spool 26 with flanges 21 slidably mounted onto stem 7. Bar 22 has a thumb grip 23 depending from its proximal end. Shaft 5 is mounted to stem 7. Cable 8 is operably attached to flange 21, so that an axial sliding movement of spool 26 will produce a similar movement of the cable 8 within shaft 5, thus opening and closing the cups 15 and 16.

Needle 11 is positioned inside the closed cups and is exposed when the Cups are open. The opposing edges of the cups are sharpened. When the biopsy forceps are in use the operator opens and closes cups 15 and 16 by sliding spool 26 on stem 7. As the cups are moved into position relative to the tissue to be sampled, the centrally positioned needle penetrates the tissue. The sharpened edges of the cups engage the tissue and sever a small sample, which is retained in the cavity formed by the closed cups. Holes 12 are commonly provided in the cups 15 and 16 to permit fluid to drain from the cups.

During most procedures two or more biopsies are taken and stacked on the needle, thereby allowing multiple biopsies to be taken in a single pass. As explained above, a tissue sample can be difficult to dislodge from its position on the needle 11 and a small sharp implement, such as another needle or a toothpick, must be manually applied by medical personnel to pry the sample from the distal end of the needle. The present invention provides a significant improvement to the safety of personnel who are otherwise exposed to the risk of being stuck by the needle or spike or being cut by the sharp edges of the cups all of which are contaminated with the bodily fluids of the patient from whom the samples were removed.

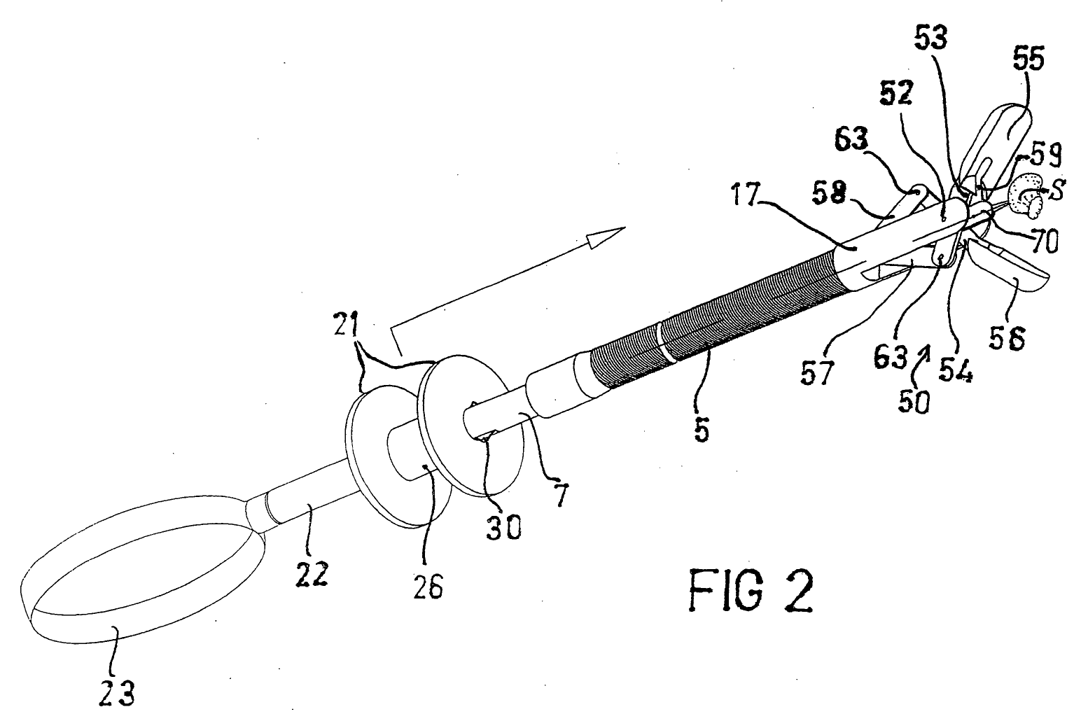

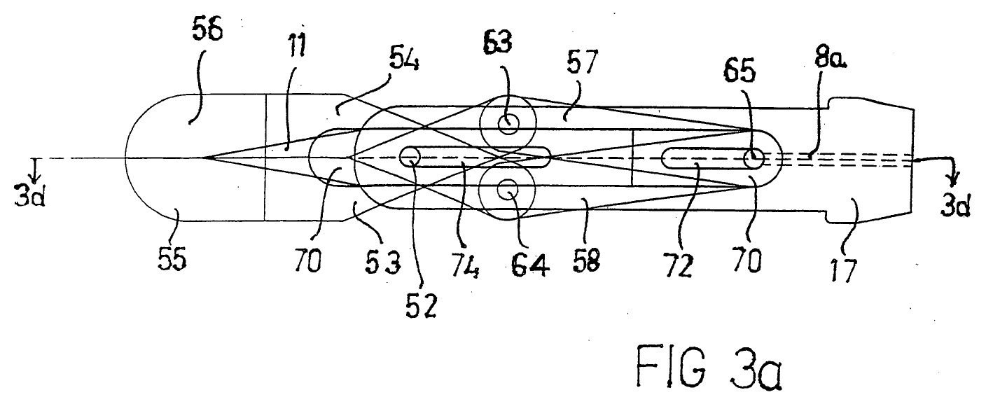

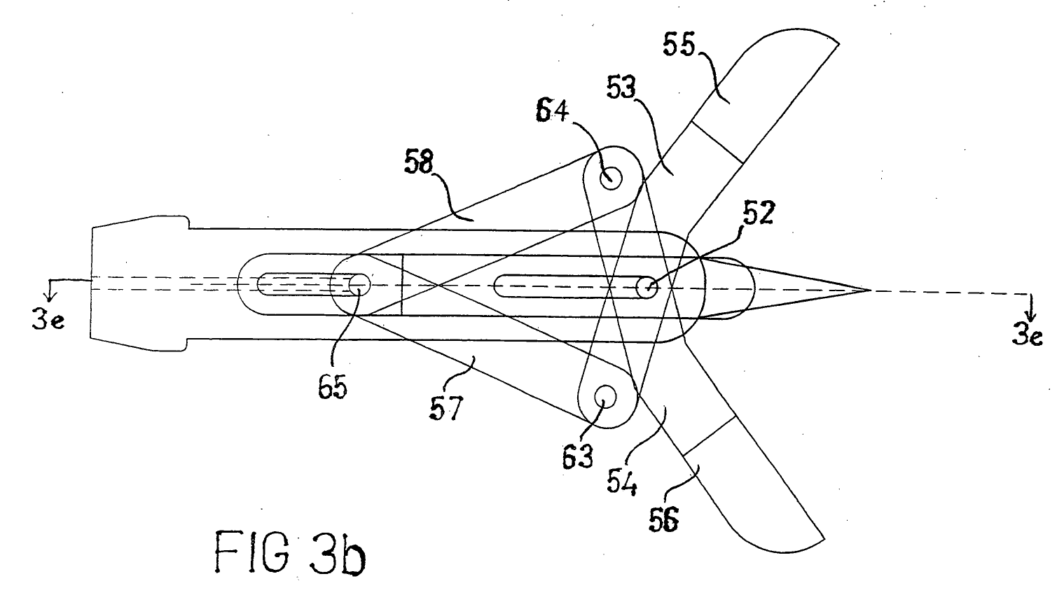

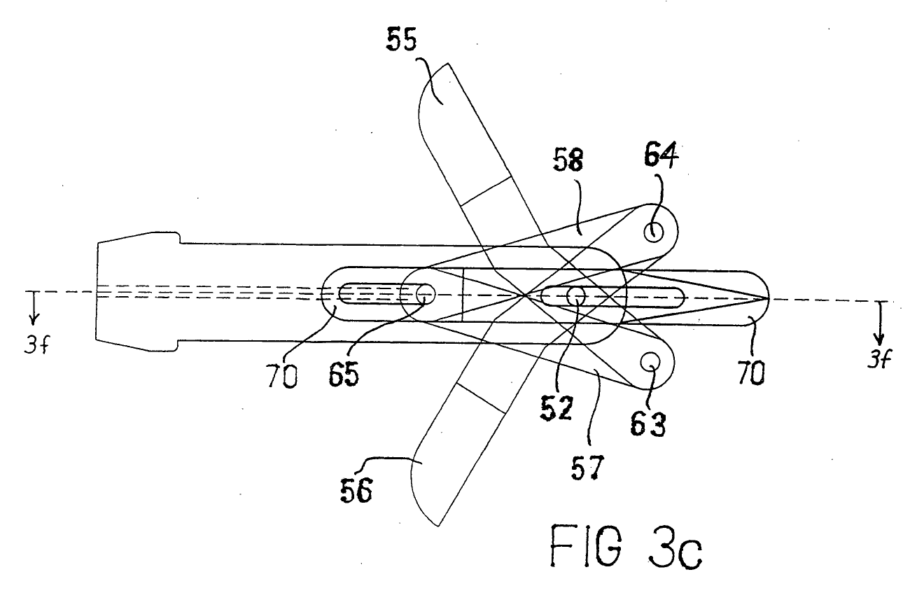

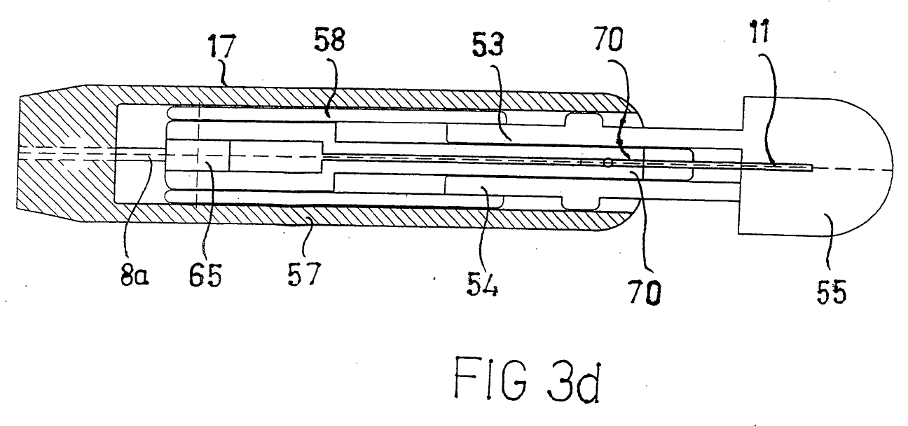

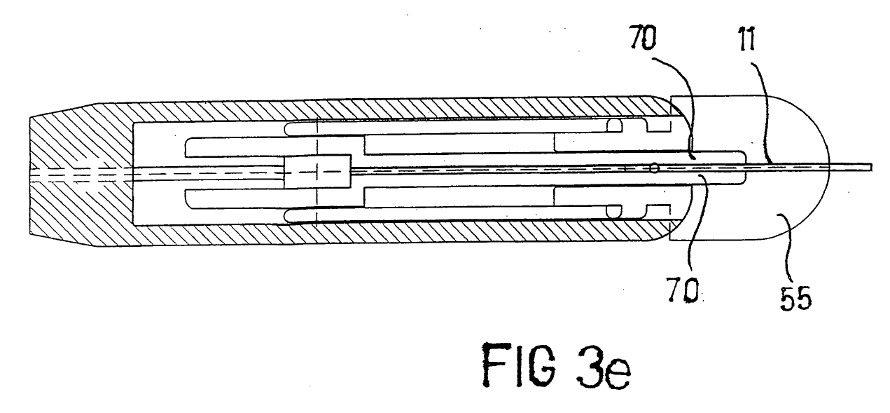

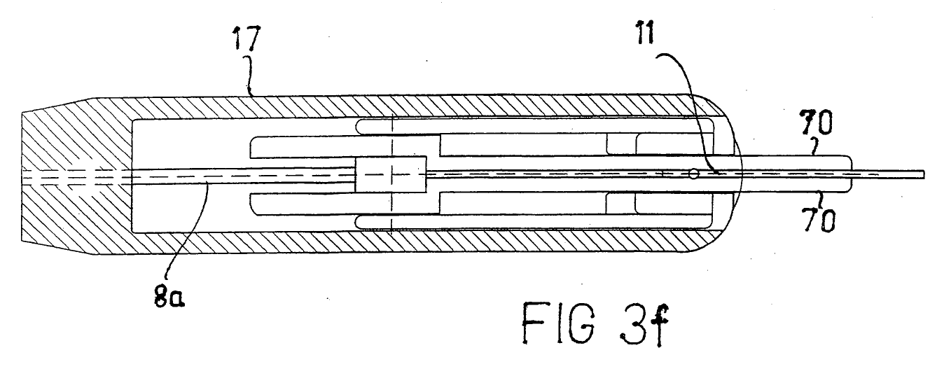

Referring now to FIGS. 2-7, one embodiment of the improved tissue sample ejection mechanism 50 of the present invention for use in needle biopsy forceps is schematically illustrated. With reference to FIG. 3a, there is illustrated the distal end of the forceps assembly that includes a stationary sample retaining spike 11 inside closed cups 55 and 56. Scissor arms or levers 53, 57 and 54, 58 are pivotally mounted at pivot pins 52, 63, 64 and 65 and operably joined to open and close cups 55, 56 in response to movement of rod 8a as actuated by thumb ring 23.

One embodiment of the present invention is configured for use with conventional endoscopes having working channels of 2.8 mm or 3.2 mm and a length of 230 cm. The cables utilized for controlling the movement of the various elements are 0.010 inches in diameter. In one embodiment, the cable or cables pass through a support tube of thin walled polymeric material having an outside diameter of 0.023 inches, which reduces frictional effects inside the spirally-wound wire comprising shaft 5 extending from the control means 20 at the proximal end.

As illustrated in FIG. 3a, the proximal ends of the levers 53 and 54 are pivotally attached to the distal ends of the scissor links 57 and 58, respectively, by pins 63 and 64, respectively. It will be understood that by sliding the cable 8 in the distal direction relative to the shaft 5, the distance between the pivot pins 52 and 65 is reduced, toggling the pivot pins 63 and 64 outward. The resulting pivoting motion of the scissors links or levers 53, 54, 57 and 58 opens the cups 55 and 56.

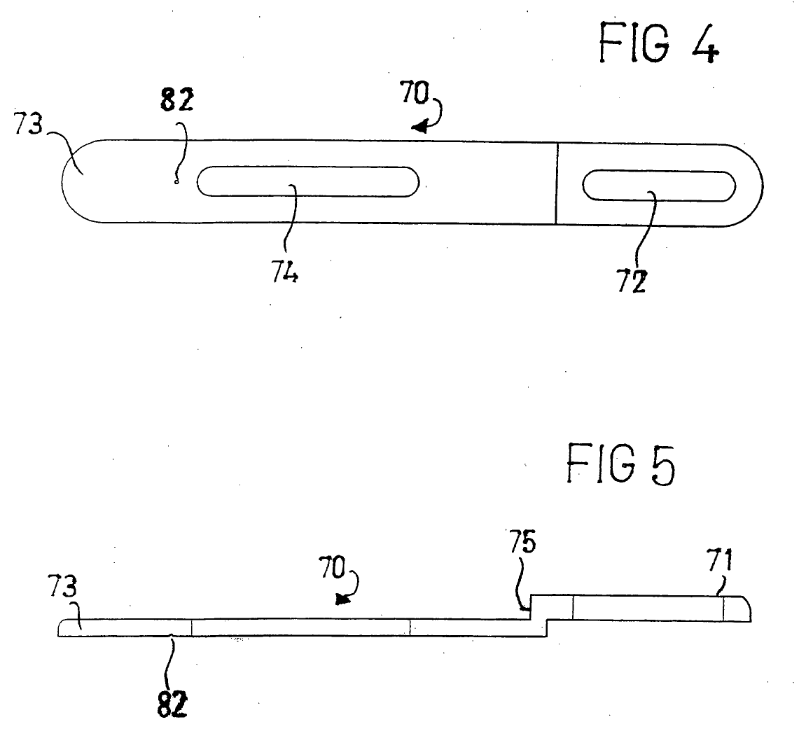

Sliding activator plates 70, also illustrated in FIGS. 4 and 5, are slidably mounted on either side of fixed spike 11, and its backing plate portion. As shown in FIG. 4, plates 70 are each provided with a proximal slot 72 and a distal slot 74 for receiving pivot posts or pins 65 and 52, respectively, said pins being shown in FIG. 3c. Pivot post 65 is attached to rod 8a (FIG. 3d), by a yoke or L-bracket, and when pivot post 65 is advanced distally, as illustrated in FIG. 3b, it initiates movement of the cups 55 and 56 to a partially open position. As pivot post 65 continues to advance distally, the actuator plates 70 (FIG. 3c) advance parallel to the longitudinal axis of spike 11 (FIG. 3d) until, as shown in FIG. 3c, the distal end portions of plates 70 are positioned at either side of the tip of spike 11.

As will be apparent to one skilled in the art, certain elements connected to the distal end of actuating cable or cables 8 may be formed from the flattened end(s) thereof, and therefore may be integral with the cable. Alternatively, the elements may be fabricated as separate elements and thereafter welded or otherwise secured to the end of the cable(s).

Thus, in this embodiment, distal end portions 73 of plates 70 (FIG. 4) provide sample contacting surfaces to controllably contact and slide a severed and captured sample off the tip end of spike 11 and into a receiving container.

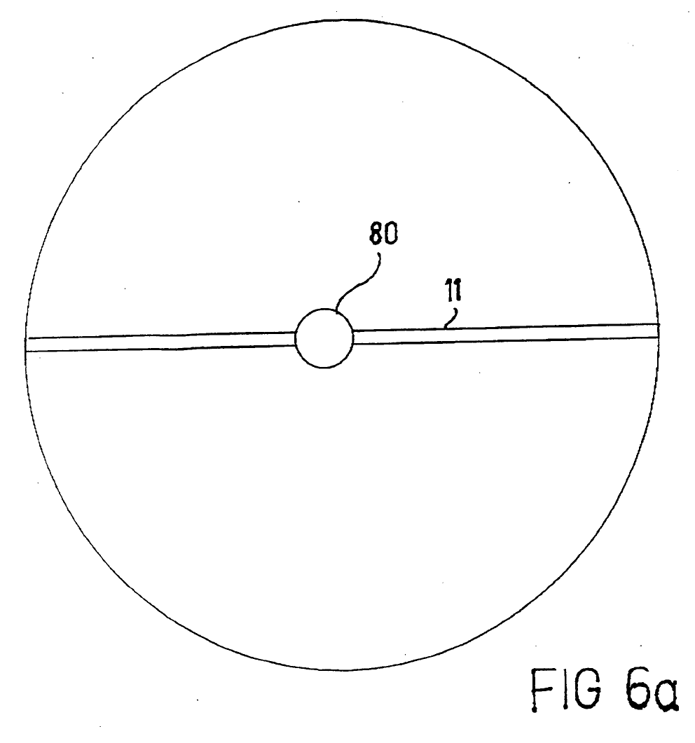

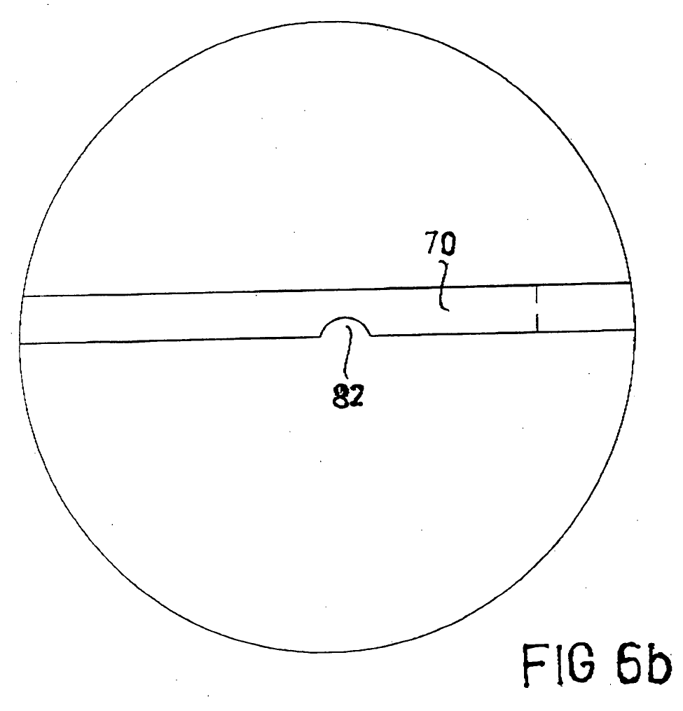

In a yet another embodiment of this aspect of the apparatus, an engagement mechanism is provided to limit the relative movement between the spike 11 and sliding plates 70. This restraining engagement mechanism prevents inadvertent relative movement between needle 11 and tissue sample contacting surfaces 73 so that the sample is not pushed off the biopsy forceps before the operator is ready to place the sample into the preservative container. The restraining engagement mechanism may be configured to include a projecting ball or sphere in the central portion of the spike and a corresponding detent or recess in one or both adjacent side plates 70. One suitable configuration is schematically illustrated in the exploded detail of FIGS. 6a and 6b wherein a ball 80 is positioned in spike 11 to be received in a corresponding recess 82 in plate 70 to thereby stop the advance of said plate. Alternative configurations, e.g., a dimpled projection and a matching detent or recess respectively provided in needle 11 and plate 70, or in another stationary member, can be utilized to stop the relative movement at a predetermined position. The force required to reverse the position of the plates 70 can be predetermined to assure smooth operation by the user.

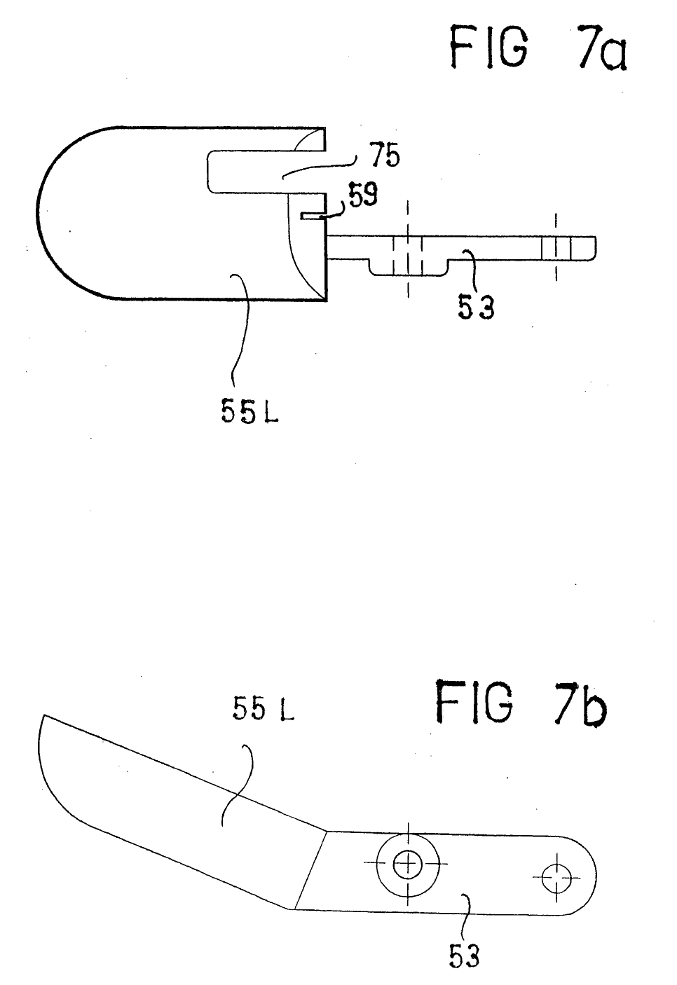

As illustrated in FIG. 2, cups 55 and 56 are provided with a groove 59 designated to receive the shaft or blade of the spike 11. It will also be understood that a smaller notch or groove opening can be provided for a needle. If a larger cup is to be utilized with the forceps, an additional and/or wider opening may be required to accommodate the scissor arms when the cups are extended to open beyond 180 degrees. A suitable configuration is illustrated in FIGS. 7a and 7b for a larger cup 55L having opening 75 adapted to receive the scissor arms.

In accordance with one embodiment of the present invention illustrated in FIG. 2, a resilient stop element 30 is provided in or proximate the handle mechanism distal to spool 26, the stop element being configured to limit forward movement of said spool. In addition, said stop element provides a mechanism, to releasably lock spool 26 in the forward position. When spool 26 is pushed, rod 8a (FIG. 3a) is moved distally. Since rod 8a is connected to moving pivot pin 65, shifting of rod 8a moves the scissor links 53, 57, 54, 58, thereby opening the cups 55, 56. The plates 70 stay in place because of the proximal hole 74 in them. When a tissue biopsy is to be obtained, the movement of sliding spool 26 is limited by resilient stop element 30, which may take the form of a proximal spring. A biopsy sample S is obtained by closing the cups 55, 56 to sever the tissue and subsequently moving the cups away from the biopsy site. Additional biopsy samples may be obtained and stacked one against the other along needle 11.

After the instrument is removed from the endoscope's working channel, the specimen is ejected into preservative solution by pushing the thumb ring 23 with enough force to overcome the biasing force of resilient stop element 30. When this is done, rod 8a begins to move the plates 70 and therefore contacting surfaces 73 distally. The cups 55, 56 open to the ejection position while the contacting surfaces 73 of plates 70 slide in the distal direction alongside needle 11 to push the specimen off the needle.

The spatial relationship of the elements is fully shown in FIGS. 3d, 3e and 3f, said figures being sectional views taken along section lines 3d-3d, 3e-3e, and 3f-3f in FIGS. 3a, 3b and 3c, respectively. As shown in FIG. 3a, levers 57 and 58 are offset from the axial centerline of cups 55 and 56. In this embodiment, scissor linkages 53 and 54 are positioned between levers 57 and 58 and opposing cups 55 and 56, and pivot about pins 63 and 64. Pivot pin 65 moves tissue ejection elements or plates 70 alternately in a distal and proximal direction, pursuant to the direction of motion of rod, wire or cable 8a. Other arrangements will be apparent to those skilled in the art for producing the required movement of the sliding plates 70 from their proximal to distal position when the cups 55 and 56 are moved to the fully opened position for relative movement of the needle and contacting surface to discharge the tissue sample.

A mechanical or electro-mechanical retraction means can be utilized to move needle 11 relative to the sample contacting Surfaces 73. A biasing spring, for example, can provide the force to produce the relative movement. The biasing spring (not shown) can be operated in the compression or the expansion mode, the device preferably being brought into an “armed” or biased position prior to use of the apparatus, and most preferably, before the forceps are placed in the working channel of the endoscope. This design and mode of operation enables the operator to prepare the forceps and check its operational characteristics prior to initiation of the procedure. It also minimizes the number of manual steps required to deposit the tissue samples into the preservative container.

Other alternatives for controlling the reciprocating movement of axial rod 8a and distal needle 11 include hydraulic and or pneumatic cylinders or pistons (not shown). Small pumps and/or pressure tanks may be utilized to provide the pressurized fluid. Such devices are in common and long-standing use, and disclosures of suitable pneumatic systems are to be found in the prior art.

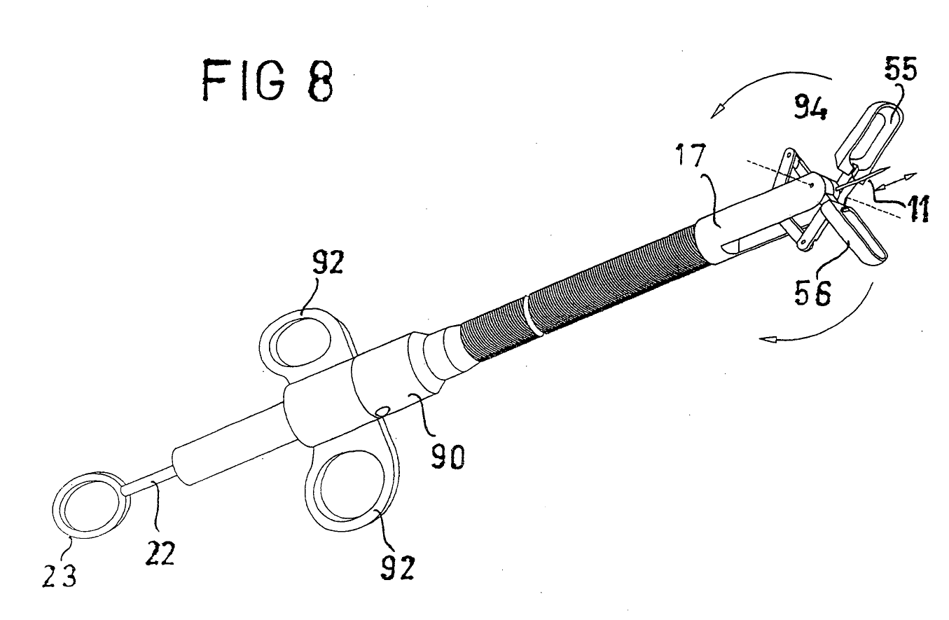

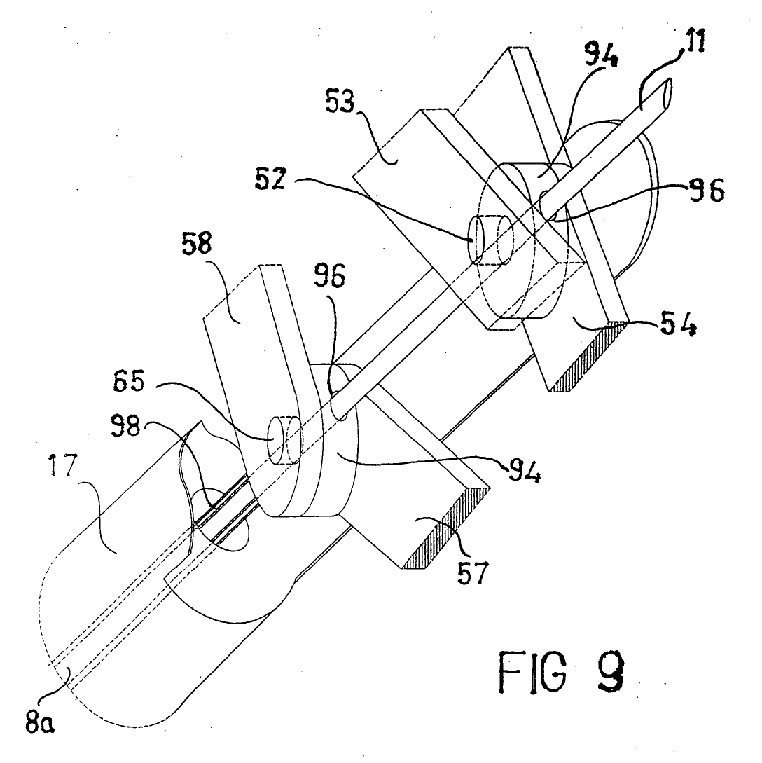

Another embodiment in accordance with the present invention is illustrated in FIG. 8 wherein the forceps' handle mechanism includes a body 90, projecting finger grips 92 and thumb piece 23. As best shown in FIG. 9, this embodiment includes a needle 11 that passes through by-pass members 94 located at pivot pins 52 and 65 described above. By-pass members 94 are provided with respective channels 96 that are traversed by the needle 11. Needle 11 is operably coupled proximally rod 8a by an appropriate friction or mechanical attachment fitting 98.

When needle 11 is withdrawn proximally, a distal face of the distal by-pass member 94 and adjacent scissor arms 53 and 54 form a sample contacting and tissue ejection surface, thereby causing sample to be moved down the needle and dislodged therefrom.

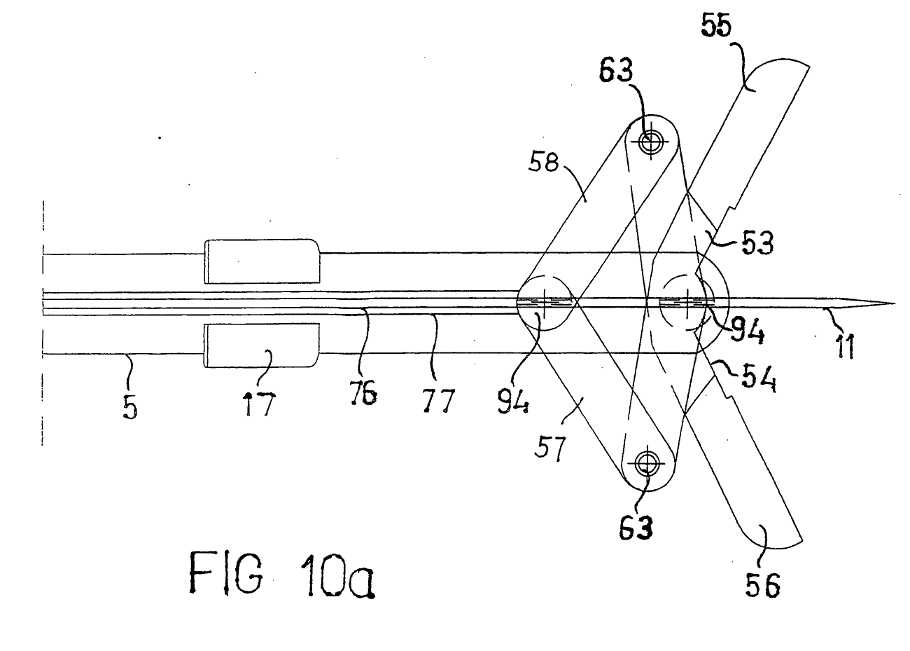

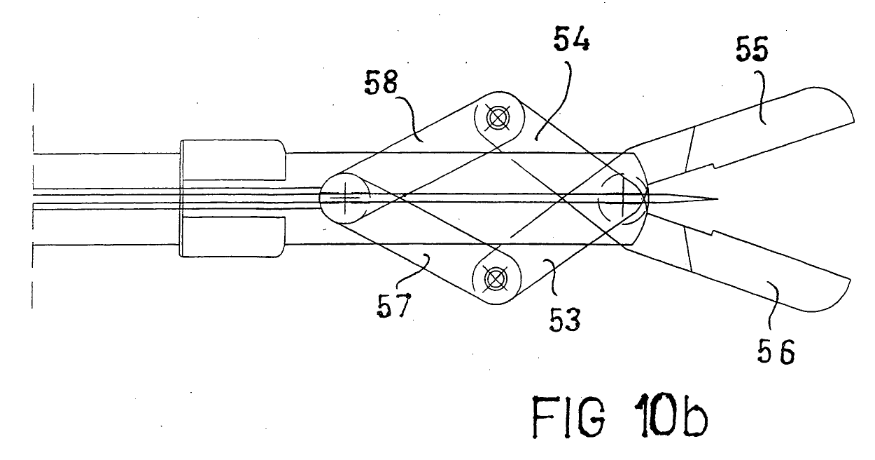

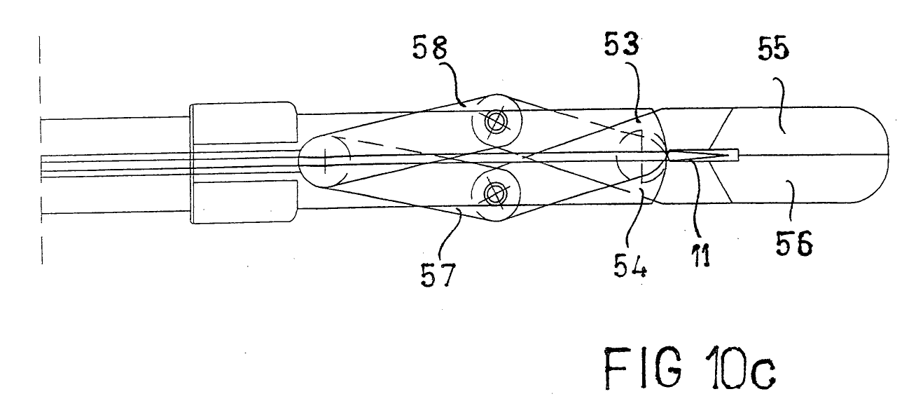

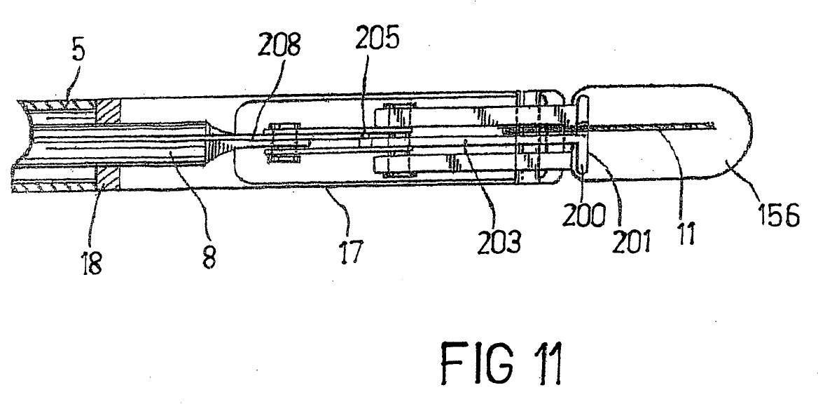

In a further embodiment of the invention illustrated in FIGS. 10a, 10b and 10c, needle 11 is configured to be mounted for reciprocal movement in response to the movement of thumb ring 23. As shown in the series of section views depicted in FIGS. 10a, 10b and 10c, a first wire (not separately designated) terminates in a hollow cylinder 77 that is operably connected to the proximal end of the proximal scissor links 57 and 58 to operate the scissor links 53, 57, 354, 58 to alternately open and close cups 55, 56. The use of cylinder 77 permits a pin 76 disposed therein to slide in a longitudinal direction freely in response to second cable 8a (see prior figures) operably attached to thumb ring 23, thereby advancing and retracting the needle. As needle 11 is withdrawn proximally, any tissue sample mounted thereon will be engaged by a sample contacting and tissue ejecting surface formed by the leading edges of open arms 53, 54 and the distal by-pass member 94. Other arrangements of the elements can be selected by one of ordinary skill in the art to effect the same resulting relative movement. For example, the distal by-pass member 94 may be disposed proximate the outer surface of one of the cup-supporting levers or scissor links 53, 54. The functional relationship of pin 76 and lumen 77 may also be reversed, substituting other obvious mechanical equivalents in their place.

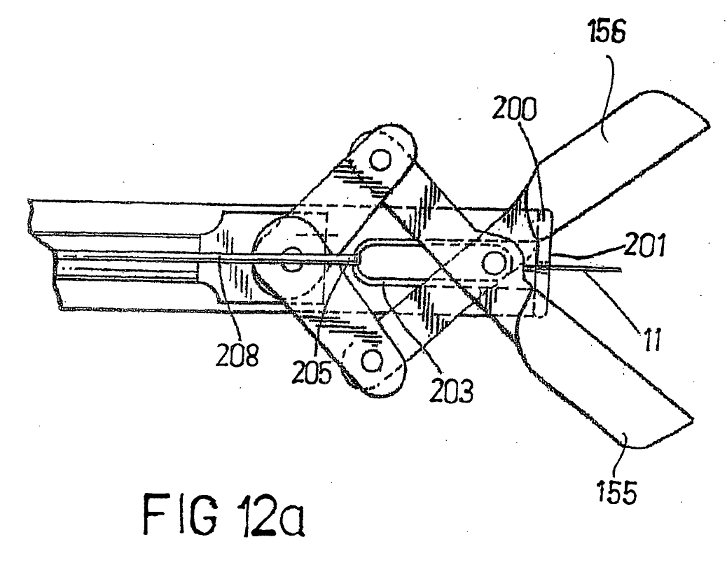

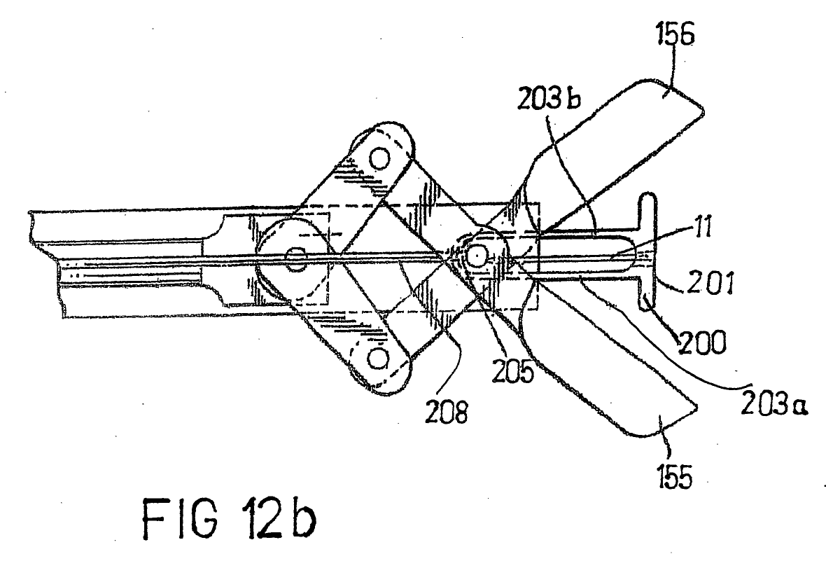

A further embodiment in accordance with the present invention is depicted in FIGS. 11, 12a and 12b. In this embodiment, a tissue ejection plate 200 is operatively connected with the distal end of a second wire, rod or cable 208 which is movably disposed in shaft 5 adjacent and parallel to cable 8. As shown in FIGS. 12a and 12b, ejection plate 200 is generally T-shaped in cross section and has a generally circular or oval top sample contacting surface 201, said surface fitting within a proximal end of tissue severing and capture cups 155, 156 when said cups are in a closed position. Joined to, or formed integrally with an underside of contacting surface 201 is a leg member 203 comprised of a pair of elements 203a and 203b joined at their proximal ends.

Wire 208 is secured to the proximal end of leg 203 at 205. The proximal end of wire 208 is secured to a manual actuator at the proximal end of the shaft 5. An ejection mechanism for shifting ejection plate 200 can comprise a separate finger or thumb grip having the configuration of thumb grip 23 shown in FIG. 7. Alternatively, wire 208 can be slidably affixed to spool 26 (FIGS. 1, 2), enabling wire 208 to be advanced in the distal direction after cups 155 and 156 have been fully opened and the release mechanism activated.

Wire 208 with ejection plate 200 may be used in conjunction with conventional biopsy forceps depicted in FIG. 1. Distal movement of wire 208 moves plate 200 in a distal direction, as shown in FIG. 12b. A tissue sample on needle 11 would thus be moved distally by contacting surface 201 of tissue ejection plate 200.

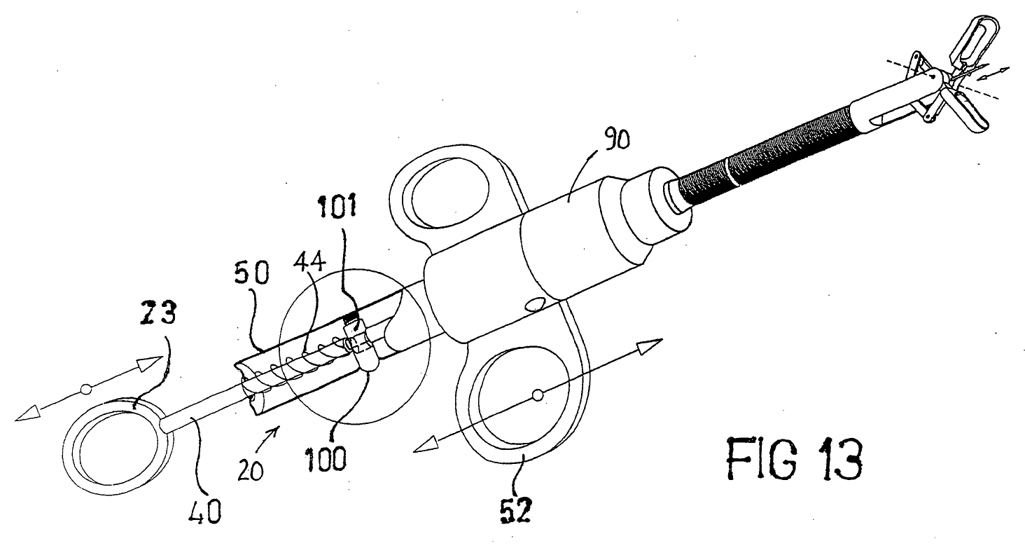

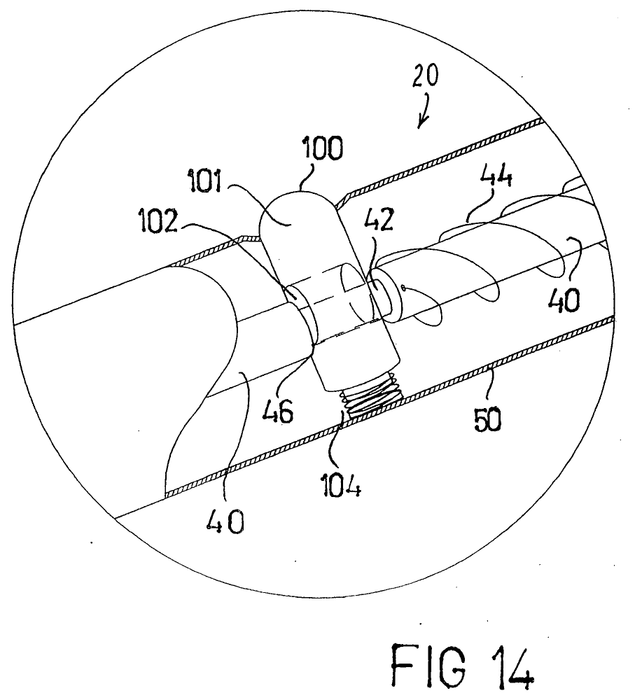

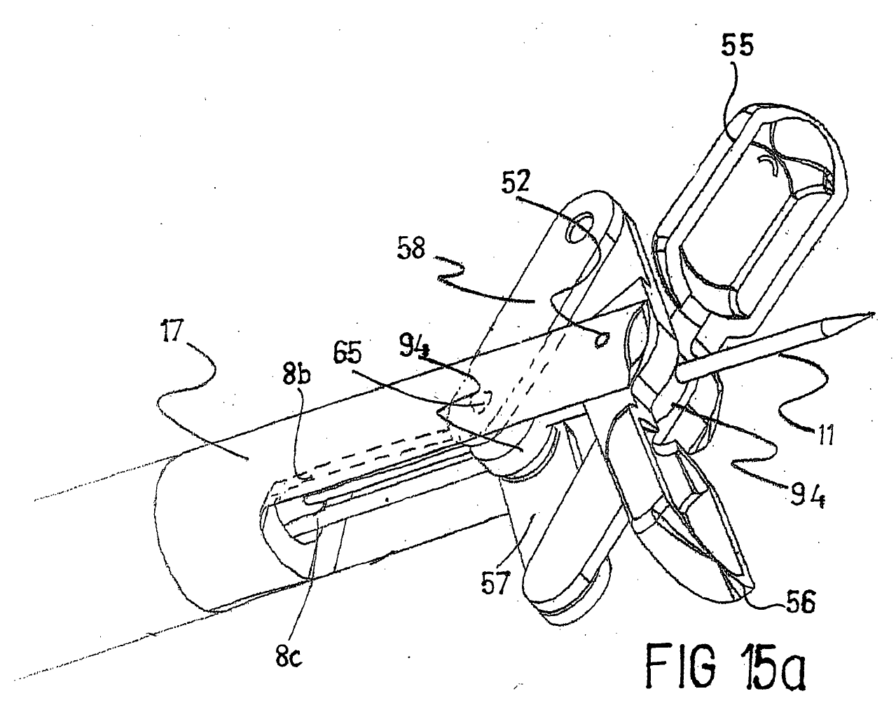

With reference to FIGS. 13 and 14, an embodiment comprises a mechanical spring-biased needle-retracting tissue-ejection mechanism activated under the control of a push-button 100 disposed on a barrel or housing 50 of a handle mechanism 20. Referring to FIG. 13 and to an exploded view depicted in FIG. 14, an axial rod 40 is provided with a slot or grooved portion 42 of a smaller diameter, said grooved portion being slidably disposed inside a channel 102 configured inside a depending shaft 101 of push-button 100. A push-button biasing spring 104 urges a lower portion of the shaft 101 against a shoulder 46 of rod 40. Opposing ends of a rod biasing spring 44 are secured to rod 40 and to housing 50, so that when the components are in the armed position illustrated in FIG. 13, spring 44 is extended. When a force F is applied to depress button 100, rod 40 moves proximally, thereby retracting the needle and dislodging tissue disposed on said needle.

As will be apparent to one of ordinary skill in the art, the biasing spring 44 maybe mounted for compression in the armed state on the opposite side of groove 42. The compression spring can itself be mounted in a groove in rod 40 and retained by a collet or other mechanical fastener (not shown).

Before inserting the biopsy forceps into the working channel of the endoscope, the needle is locked into place by pushing the thumb ring 23 distally. This action extends spring 44 causing the spring-biased push button 100 to snap into place. The forceps assembly is then inserted into the endoscope working channel.

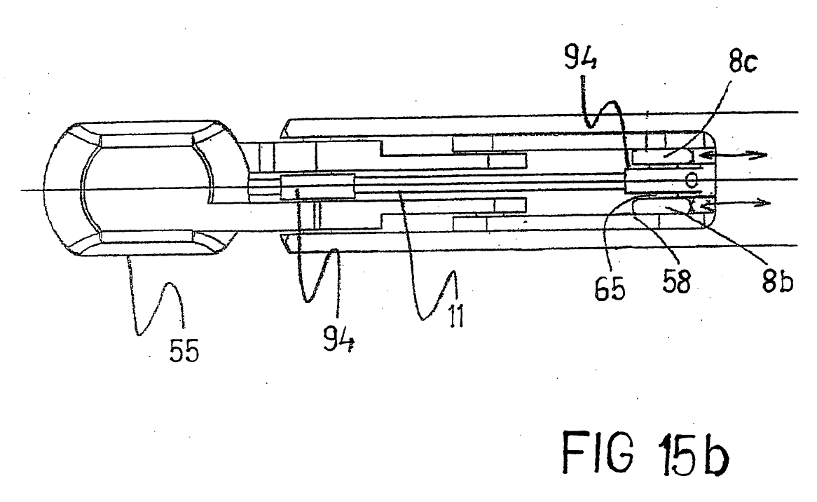

A further embodiment of the invention is illustrated in FIGS. 15a and 15b, wherein the proximal ends of scissor links 57 and 58 are pivotally mounted on proximal by-pass element 94 wherein needle 11 is slidably disposed. A pair of wires, rods, or cables 8b, 8c is operably connected to a handle mechanism such as that depicted in FIG. 8. The respective distal ends of wires 8b, 8c are secured to transverse pivot pin 65, said pivot pin also being operably coupled to scissor arms or links 57 and 58. As best shown in the partial top view of FIG. 15b, a distal movement of wires 8b, 8c causes pivot pin 65 to move in the corresponding direction thereby causing the scissor mechanism to open opposing cups 55 and 56, and exposing needle 11 bringing about sample dislodgement.

When wires 8b, 8c are moved proximally, cups 55 and 56 close. This particular two-wire design provides a direct positive control over the movement of the scissor mechanism and has the effect of providing dual activation means. As will be apparent to one of ordinary skill in the art, the use of a pair of parallel wires 8b, 8c such as is described above also serves to enhance the reliability and operability of the biopsy forceps assembly.

Before inserting the device of FIGS. 13, 14, 15a, 15b into the working channel of the endoscope it is necessary to lock the movable needle 11 in place. This is accomplished by moving the thumb ring 23 distally to compress (or stretch) a spring located in the handle portion. As described above, button 100 is shifted in a transverse direction during the distal motion of needle 11. Button 100 snaps into slot or grooved portion 42 and thereby limits the extension of needle 11 at the distal end of the instrument. If a support tube (not shown) is used to facilitate movement within the hollow flexible shaft member 5, that tube moves with the wire or cable as it is extended. The wire and the support tube, if any, can be secured to thumb ring 23 by any conventional means, e.g., adhesive.

After the endoscope (not shown) has been inserted into the patient and a biopsy site located, for instance, through optical components of the endoscope, the forceps instrument is advanced through the working channel of the endoscope until the jaws or Cups 55 and 56 extend from the distal end of the working channel. The handle actuators are then manipulated to open the jaws or cups 55, 56. Subsequently, the forceps instrument is moved in the distal direction into a target tissue mass so that the needle 11 penetrates the mass. Cups 55 and 56 are actuated to close in the tissue mass and thereby sever and capture a portion thereof, the captured tissue sample being impaled on the distal end of needle 11. Cups 55 and 56 are pivoted into an opened configuration and subsequently into a closed configuration by shifting drive wires 8b, 8c each of which may be surrounded by a support tube. The support tubes move with the drive wires 8b, 8c during actuation. Drive wires 8b, 8c may be attached to slider 90 by wire pins (not shown) of the same material as the slider which are press fit into the slider and pinch the drive wires into place. This connection is optimally accomplished during final assembly to ensure that the proper tension and tolerances are achieved for opening and closing the cups.

In addition to the holes (not shown) for the mating wire pins, the slider 90 has a slotted access opening (not shown) for tensioning of the wires with a small hand tool before they are secured in place. This access slot also allows for positioning and securing the movable needle wire into thumb ring 23. Wires 8b, 8c are tensioned in place as the wire pins (not shown) are inserted securely into the slider 90.

The button assembly 100 et seq. is retained in slot or grooved portion 42 in rod 40 that permits a relative movement of bottom 100 and thumb ring 23 while also preventing removal of the thumb ring from the handle. Button 100 includes an integrally formed cantilevered spring 104 and is locked against the handle. The user can easily depress the exposed portion of the push button 100 and release the assembly to retract the needle 11 into the body of the instrument.

After the instrument of FIGS. 13, 14, 15a, 15b is removed from the working channel of the endoscope and positioned to place the samples into the container of preservative solution, the cups 55, 56 are opened to expose the sample and the button 100 on the handle assembly 20 is pushed to retract the needle. Pushing button 100 disengages the lower retaining portion 46 of button 100 from the thumb ring post or 40, the consequent contraction of the spring 44 causing the needle 11 to be retracted proximally into the body of the forceps and the biopsy specimens to be ejected into the preservative solution.

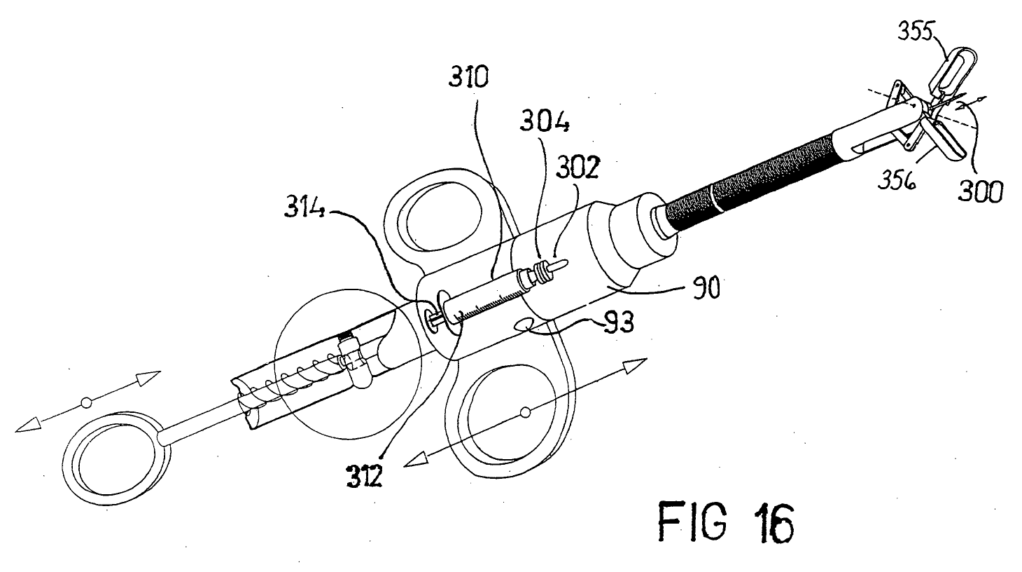

In a further embodiment illustrated in FIG. 16, a hollow needle 300 is employed. The proximal end of needle 300 is disposed in sealed fluid communication with flexible conduit 302 terminating in female fitting 304 with an internal orifice configured for receiving a standard syringe 310. Syringe 310 is conveniently secured to the forceps' handle mechanism or proximate thereto by means of spring clamps (not shown) or other appropriate releasable fastener. The apparatus of this embodiment allows the operator to inject the organ proximate to where the biopsy was obtained with tattooing ink or another biocompatible dye for the purpose of marking the site. In addition, the operator may desire to inject the area with saline solution or another biocompatible liquid for the purpose of elevating the mucosa and creating a safety cushion to perform the biopsy. As well, a dye fluid that is radio-opaque, or a fluid that contains a radioactive tracer compound may be injected through the hollow needle 300.

In the practice of the method utilizing this embodiment in accordance with the present invention, the forceps are moved proximate the portion of the tissue to be injected, the cups 55, 56 are opened to expose the hollow needle 300, and the needle is subsequently advanced to penetrate the targeted tissue. The operator then depresses a plunger 314 to eject the fluid from a barrel 312 of syringe 310 (FIG. 16). The injected fluid travels through flexible conduit 302 and is ejected from the tip of the hollow needle 300 into the target tissue. Assuming that a tissue sample is also to be collected, the cups 55 and 56 are closed to surround and sever the sample while it is positioned securely on the needle 300.

As will be clear from the above description, a plurality of samples can be collected on needle 300. Depending upon the purpose of the biopsy, one or more of the plurality of tissue sample can also be injected with fluid from syringe 310.

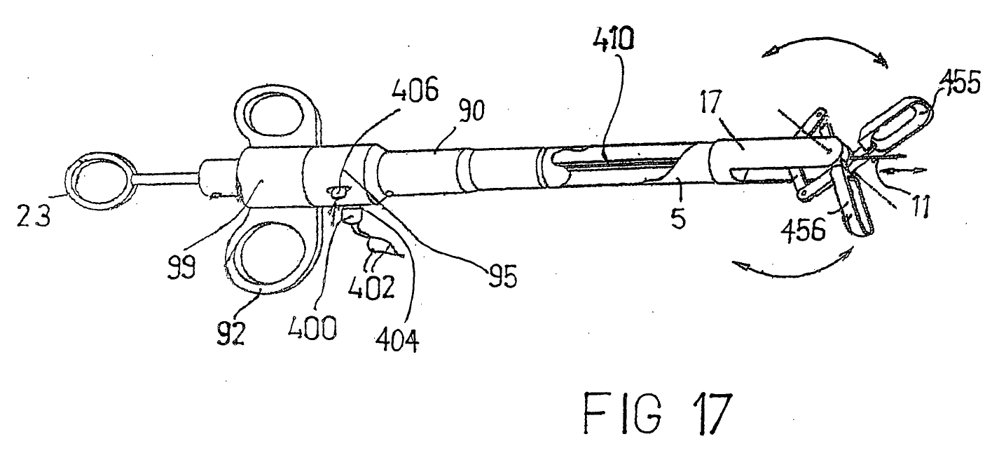

In a further embodiment illustrated in FIG. 17, a needle biopsy forceps in accordance with the present invention is capable of supplying a cauterizing current that is provided by a plug 400 with electrical conductors 402 leading to an appropriate power supply (not shown). An insulated socket 404 is fitted to forceps body 90 to matingly engage plug 400. Internal electrical conductors 410 extend through hollow flexible shaft 5 from socket 404 to electrically conductive metal cups 455, 456.

The needle 11 is electrically isolated from the conductive cups 455, 456 or is itself made from a non-conductive material, such as nylon, high density polyethylene or other suitable engineering polymer or copolymers. The needle 11 can also be insulated from the cauterizing current by the application of a conductive coating, e.g., a polytetrafluorocarbon sold under the trademark TEFLON® by the DuPont Company. The coating can be applied as a heat shrinkable web or by spraying. As will be understood by one of ordinary skill in the art, it is preferred to minimize the heat to which the recovered sample is subjected and the cups 455, 456 can be made large enough to enclose, but not contact the severed tissue sample.

In the practice of the method set forth in this embodiment in accordance with the present invention, the cups 455, 456 are opened and needle 11 is moved into position to penetrate the tissue to be sampled. The forceps instrument is then moved in the distal direction so that the needle 11 penetrates tissues to be captured. The thumb and finger grips 23, 92 are subsequently moved to clamp the cups 455, 456 around the tissue on the needle 11 and sever it from a surrounding tissue mass. Simultaneously, a switch 406 is activated to send a brief cauterizing current through cups 455, 456 into the tissue mass to thereby stop or minimize bleeding therefrom. The forceps may be manipulated to collect additional samples from the same or a different organ or withdrawn for removal of the one or more samples harvested during the procedure in accordance with the description provided above.

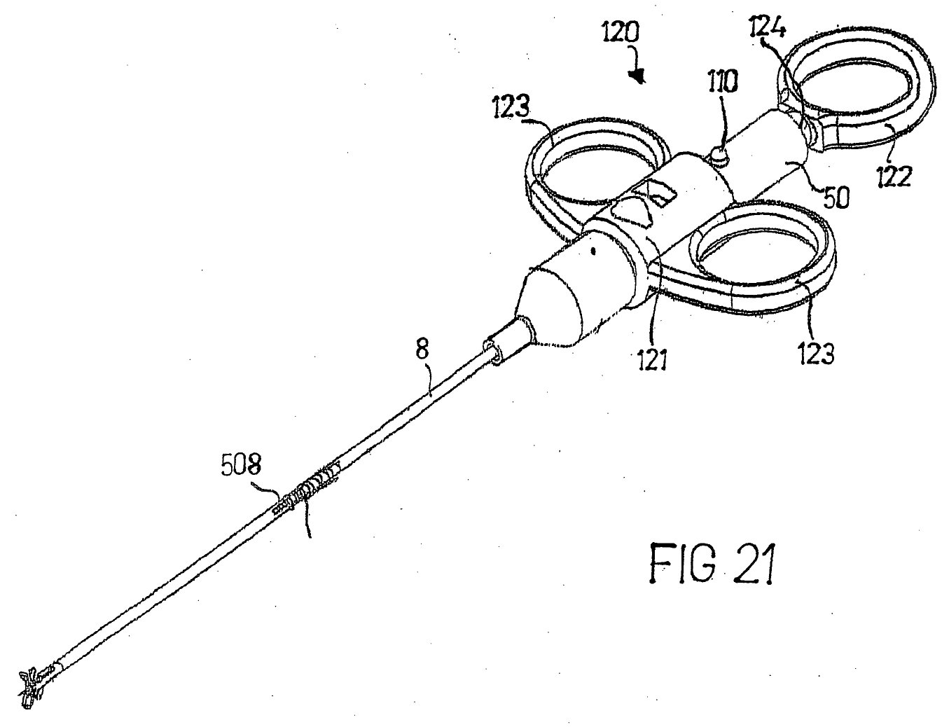

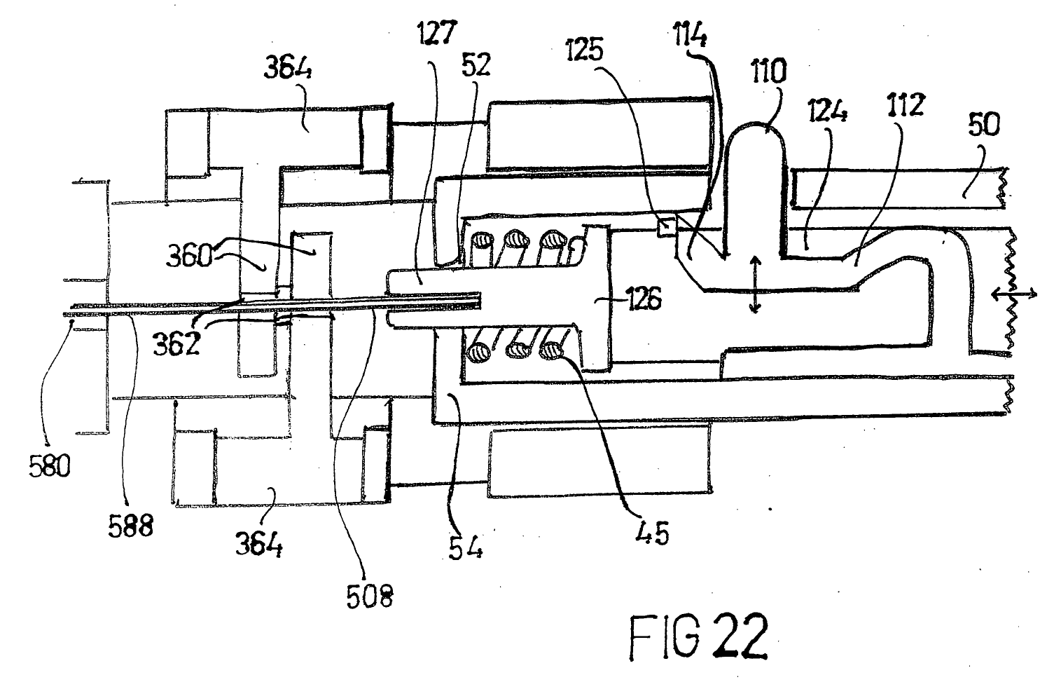

In addition to the control mechanism illustrated and described in connection with FIGS. 13 and 14, another embodiment of manual controls and releases positioned in or proximate the handle mechanism is illustrated in FIGS. 21 and 22. As will be understood from the prior description, the invention has as one principal purpose and object, namely ensuring the safety of the medical personnel using it. Because of various control systems installed at or proximate the handle mechanism, personnel may avoid having to bring their fingers into close proximity with the distal end of the instrument, thereby minimizing or eliminating the danger of incurring a puncture wound from the needle 11 or a cut from the sharpened jaws of the cups 55, 56, 455, 456 etc.

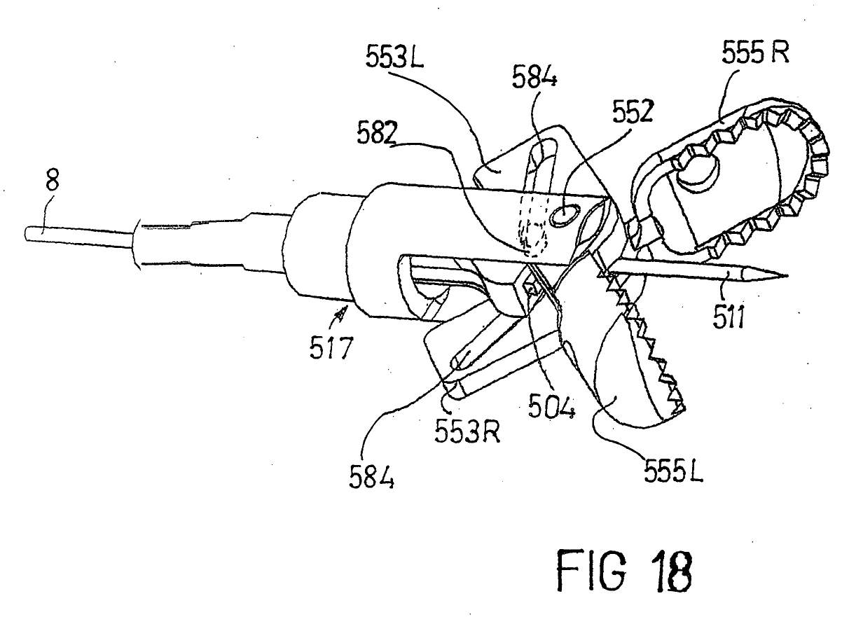

A further embodiment in accordance with the present invention is illustrated in FIGS. 18-20 and comprises a cam mechanism employed to open and close opposing biopsy-gathering cups or jaws 555R, 555L. In the embodiment illustrated, a needle 511 is integrally formed as part of a needle control assembly 500 that further includes a needle advance stop member 502 and a retraction stop member or surface 504 formed at pre-determined distances from the sharpened needle tip, and a proximally extending tail piece 506 for attaching to an axial wire 508 that controls the movement of the needle portion 511. The needle control assembly 500 is preferably formed as one piece, as by stamping from a sheet material, machining or by molding from metal or polymeric materials that are well known in the art. This unitary construction of assembly 500 provides economy in manufacture and ease of assembly.

The distal end of needle 511 may be passed through a central opening 596 in a bypass member 594 that is preferably integrally formed with opposing transverse pivot posts 552 on which the cups 555R and 555L are pivotally mounted. The ends of pivot posts 552 are retained in corresponding openings 557 at the distal ends of the opposing arms of a clevis 517.

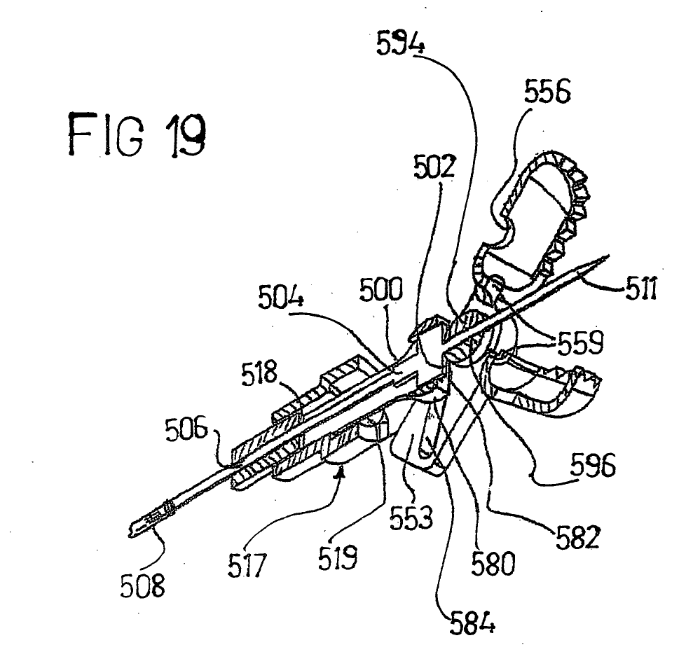

As best shown in the cross-sectional view of FIG. 19, the clevis 517 is formed with an axial opening 519 having an internal shoulder 518 set forth for the purpose of engaging a proximal surface of needle retraction stop 504 to limit the range of the movement of needle 511. As also shown in FIG. 19, the axial opening or channel 519 of the clevis 517 extends proximally to receive the needle assembly tail piece 506.

As in other embodiments discussed herein, each of the cups 555R, 555L is provided with a groove 559 to permit passage of needle 511. The cups may also be provided with at least one orifice 556 to permit the passage of fluid.

The opening and closing of cups 555r, 555L is controlled by cam and follower means. A pair of cams in the form of fixed posts 582 extends transversely from a drive member 580 and contact cam follower surfaces 584 formed in the respective cup arms 553. The drive member 580 slides axially through the channel or axial passageway 519 in clevis 517. The cups are opened by moving the drive member 580 distally causing the cam follower surfaces 584 to move away from the axis of the instrument; moving the drive member 580 proximally brings the cup arms 553 toward the axis in response to the movement of the cam follower surfaces 584 against the proximal movement of cam post 582. As will be apparent to one of ordinary skill in the art, the maximum extent to which the cups open is a function of the length of the cam follower opening and its angle with respect to the axis of the instrument.

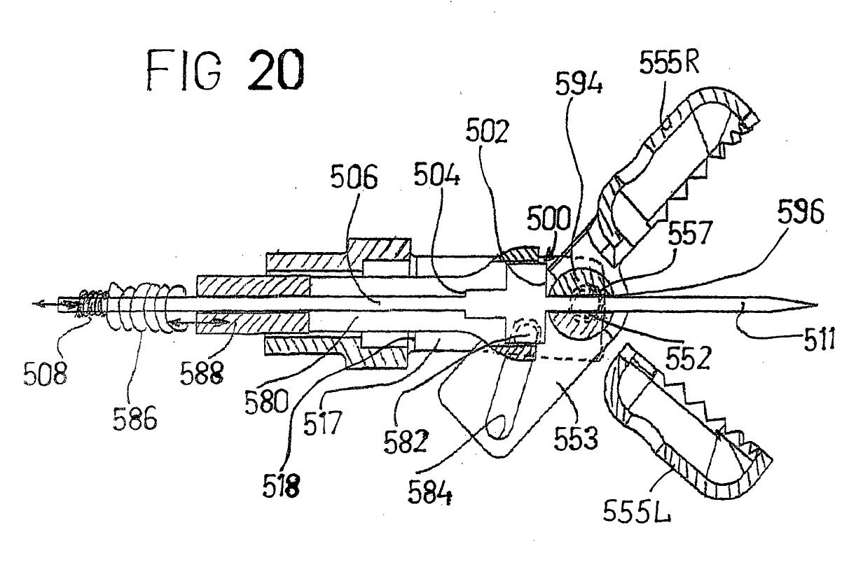

Referring to FIG. 20, the axial movement of the drive member 580 is preferably controlled by a drive tube 586 in the form of a coiled wire cable that is co-axial with the needle assembly control cable 508, both of which extend proximally to control elements in the handle assembly (reference numeral 20 in other drawing figures). In one specific embodiment, the drive tube 586 terminates at, and is permanently attached to a slide with finger grips or a spool member of the type known to the art. See, for example, FIGS. 1 and 15. Other means of controlling the movement of the cups 553, including those described above in connection with other embodiments of the invention can also be employed without affecting the utility of the overall advantages of the invention.

With continuing reference to FIG. 20, the needle assembly is slidably retained by its passage through central channel 596 in bypass member 594. The bypass member 594 also serves to determine the distal movement of the needle 511 by engaging the distal end or surface (not separately designated) of the needle stop member 502.

As best shown in the cross-sectional side view of FIG. 20, the drive member 580 is provided with opposing cam posts 582 that engage cam follower surfaces 584 in the cup arms 553. As will be explained in further detail below, when the drive member and its associated posts 582 are moved from a first position to a second position, the cam action causes the cups 555 to move from a closed to a fully-opened position. As will be understood by one of ordinary skill in the art, other cam follower surface arrangements can be provided to achieve the same result. For example, curvilinear and dual angled cam follower surfaces can be provided to change the rate of movement and forces applied to the cups 555r, 555L for severing tissue samples.

The movement of the needle 511 and the cups 555R, 555L can be controlled independently by a thumb ring and slide linking member in a handle assembly, respectively, in a manner similar to that described above in connection with the method of operation of other embodiments.

Referring to FIG. 21, as in previously described embodiments, a thumb ring 122 is joined to the tail piece 506 (FIG. 20) of needle control assembly 500 by a flexible wire, wire tube or coil that extends through the interior of exterior cable 8. The thumb ring 122 is first joined to a shaft 124 which slides in barrel or housing 50. A push button 110 extends through barrel 50 and engages a needle release mechanism that, in turn, is joined to the co-axial flexible needle tube or coil 508.

With continuing reference to FIG. 21, a sliding spool or sleeve 121 with finger grips 123 is also mounted on the handle 52. The sliding sleeve 121 is attached via one or more wires or a co-axial tube whose axial movement opens and closes the cups via scissor mechanism described above, or by other means which will be described below.

Referring now to the cross-sectional view of FIG. 22, the needle control locking and release mechanism will be further described. The push button 110 is attached to a generally C-shaped resilient arm 112 that provides a biasing force that resist a downward force on button 110, thereby causing lock arm 114 to engage the proximal surface rim lock member 122 which is formed as part of thumb ring shaft 120. The tip 122 passes through close-fitting orifice 52 in barrel 50 and receives needle control wire 508 in secure attachment. Tip 124 terminates in proximal flange 126 and has mounted thereon spring 45. As illustrated in FIG. 22, the thumb ring is in the distally advanced position which compresses spring 45 between the end wall 54 of barrel 50 and flange 30, while lock arm 114 retains thumb ring shaft 120 by engaging rim lock 122. This illustration thus represents the operating position of the needle during the insertion, use and withdrawal of the needle biopsy forceps.

In order to disengage the samples from needle the cups are opened by advancing the sliding forger rings distally, positioning the cups over the sample collection container and then depressing the push button 110. Spring 45 moves the flange 126 proximally producing a corresponding movement of the needle 11.

In a further preferred embodiment of the handle assembly, the movement of the wire tube 588 attached to drive 580 is also controlled by a locking mechanism. A pair of adjacent opposing clamp members 360 are provided with oversize channels 362′ to receive wire tube 588. Each clamp terminates in an external manual gripping element 364 that extends above the handle 52. Once the finger rings have been moved to open the cups, the user grips the elements 364 between thumb and forefinger and squeezes, thereby causing the clamps to move radially inward and frictionally engage the wire coil passing through the respective openings 362. Once this radial force is released, the frictional effect is dissipated and the cups can be closed by movement of the finger rings.

In a particularly preferred embodiment illustrated in FIGS. 21 and 22, a locking button is provided in the handle assembly. Before inserting the device into the working channel of the endoscope, the needle assembly 500 is locked in place with needle 511 positioned inside the cups by moving the thumb ring distally to compress spring 45 located in the handle. This action ratchets and displaces the handle as a result of the thumb ring travel; the button then snaps into the corresponding notch after the advance needle stop 502 makes contact with the distal bypass 594. The needle is in the extended position and contained within the closed cups.

The manual proximal control is preferably connected to the needle assembly by means of a flexible needle control tube, but other flexible connection means, such as a wire can also be used.

With further reference to FIG. 21, a slider positioned in the handle is connected by a flexible drive tube 588 to the drive member 580.

After one or more biopsy samples are mounted on the needle and severed, the cups are returned to the closed position and the assembly is withdrawn from the working channel of the endoscope. Once removed, the cups are positioned over a container of preservative solution, whereupon they are opened and the needle release button is depressed to cause the biasing spring to retract the needle, thereby dislodging the biopsy samples by contact with the distal bypass member 594. The engagement of the proximal portion of the needle stop with the interior shoulder 518 of the clevis 517 prevents the needle from passing through the channel 596 in the bypass member.

Other configurations of the safety ejection needle biopsy forceps within the scope of the present invention, including additional combinations of the embodiments illustrated and described above, alone or in conjunction with other features and elements known to the prior art, will be apparent to those of ordinary skill in the art. The method and apparatus of the invention is not limited for use in biopsy forceps, but can incorporated for use in other types of medical instruments in which the relative movement between a retaining needle containing one or more samples and a contacting surface is effective in dislodging samples from the needle. The scope of the invention is therefore to be determined with reference to the claims that follow.

1. A needle biopsy instrument for retrieving and ejecting a tissue sample taken from an organ, comprising:
an elongate hollow shaft member with a proximal end and a distal end;
a needle connected at least indirectly to said shaft member proximate the distal end thereof, said needle being oriented to pass through a portion of tissue to be sampled before the tissue sample is severed from the organ;
a tissue ejection element mounted at least indirectly to said shaft member at said distal end thereof; and
an ejection mechanism operatively connected to at least one of said needle and said tissue ejection element for moving said needle and said tissue ejection element relative to one another so that said tissue ejection element engages or contacts the tissue sample and the tissue sample is dislodged from a distal end of said needle.

2. The biopsy instrument of claim 1, wherein said tissue ejection element includes a sample contacting surface.

3. The biopsy instrument of claim 2, wherein the sample contacting surface comprises a portion of a pivot assembly formed by arms connected to a plurality of cups.

4. The biopsy instrument of claim 3, wherein said needle passes through a needle-receiving orifice formed in the contacting surface.

5. The biopsy instrument of claim 4, wherein the needle-receiving orifice is formed in a member that is attached to an arm connected to one of said plurality of cups.

6. The biopsy instrument of claim 1, further comprising:
first and second opposing cups pivotally attached to said distal end of said shaft member for severing the tissue sample from the organ, said opposing cups each having a needle receiving aperture;
first and second scissor links pivotally attached respectively to said first and second opposing cups,
said needle being disposed in a space formed by said opposing cups in a closed configuration or position thereof,
said tissue ejection element being a pushing member formed integrally with the scissor links, said needle having a longitudinal axis passing through a receiving orifice in the pushing member.

7. The biopsy forceps of claim 6, wherein the pushing member is moveable from a first position at a proximal end of said needle when said first and second opposing cups are in a closed position to a second position at a distal end of said needle, whereby a tissue sample disposed on said needle is displaced from the distal end of the needle in the second position.

8. The biopsy instrument of claim 1, wherein said needle is mounted to said shaft member for motion relative to said tissue ejection element, said ejection mechanism being operatively connected to said needle for shifting same in a proximal direction relative to said tissue ejection element.

9. The biopsy instrument of claim 1, wherein said tissue ejection element is mounted to said shaft member for motion relative to said needle, said ejection mechanism being operatively connected to said tissue ejection element for shifting same in a distal direction relative to said needle.

10. The biopsy instrument of claim 1, wherein said tissue ejection mechanism includes a cable or shaft member longitudinally traversing at least a portion of said shaft member and operatively connected to an actuator disposed at the proximal end of said shaft member.

11. The biopsy instrument defined in claim 1, wherein said shaft member is flexible.

12. The biopsy instrument defined in claim 1, further comprising:
a plurality of opposing cups pivotally attached to said distal end of said shaft member and moveable between an open configuration and a closed configuration for severing the tissue sample from the organ, said needle being positioned proximate to said cups and contained within said cups in the closed configuration thereof;
a first cable slidably disposed within said hollow shaft member and operably connected to said plurality of cups for moving same from said open configuration to said closed configuration;
a second cable slidably disposed within said shaft member, said ejection mechanism comprising said second cable;
first control means on said proximal end of said shaft member for moving said first cable from a first position, wherein said cups have said closed configuration, to a second position, wherein said cups are in said open configuration;
second control means on said proximal end of said shaft member for sliding said second cable to thereby produce relative movement between said tissue ejection element and said needle, whereby said tissue sample is dislodged from the distal end of the needle.

13. The biopsy instrument defined in claim 1, further comprising:
first and second opposing cups pivotally attached to said distal end of said shaft member for severing the tissue sample from the organ, said opposing cups each having a needle receiving aperture, said needle being disposed in a space formed by said opposing cups in a closed configuration thereof;
first and second scissor links pivotally attached respectively to said first and second opposing cups;
a first cable assembly slidably disposed within said shaft member and having a distal end operably coupled to said scissor links, whereby a movement of said first cable assembly in said shaft member toggles said scissor links and opens said opposing cups; and
a second cable slidably disposed within said shaft member and having a distal end operably joined to a proximal end of said needle, whereby movement of said second cable moves the distal end of said needle to a position that is proximal to said scissor links when said opposing cups are in an open configuration.

14. The biopsy forceps of claim 13, wherein the second cable passes through an orifice formed in a pivot portion of the scissor links.

15. The biopsy forceps of claim 14, wherein said first cable assembly comprises two parallel cables, the distal end of each of said parallel cables being operably connected to a portion of each of the scissor links.

16. In a needle biopsy forceps having an elongate hollow shaft member with a proximal end and a distal end, a needle for passing through a portion of tissue to be sampled, a plurality of opposing cups on the distal end of said shaft member for severing and retrieving a tissue sample, at least one first cable slidably disposed within the shaft member and operably attached to said opposing cups, and cup positioning means mounted at the proximal end of said shaft member for sliding the at least one first cable, the improvement comprising: a tissue ejection element for advancing a sample of tissue on said needle toward a distal end of said needle, and motion inducing means operatively connected to at least one of said needle and said tissue ejection element for moving said one of said needle and said tissue ejection element relative to the other of said needle and said tissue ejection element, thereby moving said sample of tissue relative to said needle.

17. The improvement of claim 16, wherein said motion inducing means includes a second cable slidably disposed within the shaft member and control means for sliding the second cable mounted on the proximal end of the shaft member.

18. The improvement of claim 17, wherein said tissue election element comprises a sample-contacting surface operably joined to a distal end of the second cable, said contacting surface configured to pass along a longitudinal axis of said needle.

19. The improvement of claim 17, wherein said second cable is operably joined to said needle to move said needle from a first position in a closed configuration of said opposing cups to a second position displaced proximally from the first position.

20. The improvement of claim 16, wherein said motion inducing means includes the at least one first cable.

21. The improvement of claim 16, wherein said tissue ejection element surrounds the needle during the relative movement.

22. The improvement of claim 16, wherein said tissue ejection element comprises a portion of pivot arms that support said opposing cups.

23. The improvement of claim 16, wherein said needle forms a distal portion of a needle assembly that further comprises an advance stop portion and a retraction stop portion for delimiting the range of axial movement of said needle relative to said tissue ejection element.

24. The improvement of claim 23, wherein said needle assembly further comprises a tail piece extending proximally from said retraction stop portion, said tail piece being operably connected to a second cable slidably disposed within said shaft member.

25. The improvement of claim 23, wherein said needle assembly is received for axially movement in a recess in a supporting clevis, said clevis being provided with means for pivotally mounting said plurality of cups for movement.

26. The improvement of claim 16, wherein said control means includes a latch for selectively preventing relative movement of the needle and the tissue ejection element from a first position for retrieving a tissue sample and a second position for pushing the tissue sample from the needle.

27. The improvement of claim 16, wherein each of said plurality of cups is slotted to receive a portion of a pivot arm when said cups are in a fully opened position during ejection of the tissue sample from the needle.

28. The improvement of claim 16, wherein said needle is hollow, further comprising a syringe operably mounted on said proximal end portion of said forceps, the proximal end of said needle being in fluid communication with an interior of the syringe, whereby activation of said syringe discharges fluid from the distal end of said needle.

29. The improvement of claim 16, which further comprises conductive means for delivering a cauterizing electrical current to said opposing cups, a switch for controlling a current flow to the conductive means and wherein said needle is non-conductive.

30. A method for retrieving a tissue sample taken from an organ through the biopsy channel of an endoscope, the method comprising:
a) providing a needle biopsy instrument comprised of:
a flexible, hollow shaft member with a proximal end and a distal end;
a needle disposed proximate the distal end of the shaft member and oriented to pass through a portion of tissue to be sampled before the tissue sample is severed from the organ;
tissue sample pushing means disposed at a proximal end of said needle for movement proximally to distally relative to said needle;
at least one cable slidably disposed within said hollow shaft member and extending from associated control means at the proximal end of the shaft member to the distal end;
whereby sliding said cable produces relative movement between said needle and said pushing means to thereby dislodge a severed tissue sample from the distal end of the needle;
b) inserting the biopsy instrument through the biopsy channel of the endoscope until a portion of the distal end of the biopsy instrument extends beyond the distal end of the endoscope;
c) positioning the distal end of the biopsy instrument adjacent a portion of tissue to be sampled;
d) moving the distal end of the biopsy instrument to cause the needle to pierce the tissue;
e) severing a portion of the tissue surrounding the needle from the organ;
f) withdrawing the biopsy instrument and tissue sample from the channel of the endoscope; and
g) moving the pushing means relative to the needle to dislodge the sample from the needle.
